(19) United States
(12) Patent Application Publication
(10) Pub. No.: US 2019/0159941 A1
(43) Pub. Date: May 30, 2019

(54) CHAFE REDUCTION OR PREVENTION APPARTUS AND METHOD

(71) Applicants: Sivan Gavish, Las Vegas, NV (US); Iddo Gavish, Las Vegas, NV (US)

(72) Inventors: Sivan Gavish, Las Vegas, NV (US); Iddo Gavish, Las Vegas, NV (US)

(21) Appl. No.: 16/206,225

(22) Filed: Nov. 30, 2018

Related U.S. Application Data
(60) Provisional application No. 62/593,064, filed on Nov. 30, 2017.

(51) Int. Cl. A61F 13/06

(52) U.S. Cl. CPC A61F 13/06

(57) ABSTRACT

Anti-chafing pads that are adherable to the skin for prevention of chafing are provided. The anti-chafing pads may be used to prevent or reduce chafing on the skin of a user.

### CROSS-REFERENCE TO RELATED APPLICATIONS

The present application claims the benefit of U.S. Provisional Patent Application No. 62/593,064 (pending), filed on Nov. 30, 2017, the entirety of which is incorporated herein by reference and made a part of the present disclosure.

### FIELD

The present disclosure relates to apparatus and methods for reducing and/or preventing chafing.

### BACKGROUND

Chafing occurs when skin frictionally interacts with (i.e., rubs against) clothing or another portion of skin. The experience of chafing can be an ongoing annoyance throughout the day, and often results in pain, rash, redness, and irritation of the skin. While chafing can occur on any portion of skin anywhere on the body, one common location of chafing is between the inner thighs, which rub together while walking for people that do not have a “thigh gap.” Such inner thigh chafing has been described by some as feeling as if their thighs are rubbing against a cheese grater.

Prior attempts to prevent chafing include: the wearing of spandex, which feels constricting to the wearer; the application of deodorant to the skin, which causes discoloration (e.g., whitening) of clothes and is prone to lose effectiveness over time; and the wearing of thigh bands, such as BANDELETTES®, which include a lace material that does not sit on the thighs. Other prior attempts at preventing chafing include the application of body powder (e.g., GOLD BOND®) and glide creams to the skin. Some women have even tried wearing “boy shorts” or even men's “boxer briefs” in an attempt to prevent chafing.

### BRIEF SUMMARY

In one aspect, the present disclosure provides for an anti-chafing pad. The anti-chafing pad includes a protective layer and an adhesive layer on the protective layer. The adhesive layer is adherable to skin.

Another aspect of the present disclosure provides for an anti-chafing pad that includes a protective layer, an adhesive layer on the protective layer, and a cover layer disposed on the adhesive layer. The adhesive layer is positioned between the cover layer and the protective layer. The cover layer is selectively removable from the adhesive layer, and the adhesive layer is adherable to skin. The anti-chafing pad, when worn on skin, prevents or reduces chafing of the skin.

Another aspect of the present disclosure provides for a method of preventing or reducing chafing on skin of a user. The method includes providing first and second anti-chafing pads. Each anti-chafing pad includes a protective layer, an adhesive layer on the protective layer, and a cover layer disposed on the adhesive layer. The adhesive layer is positioned between the cover layer and the protective layer. The cover layer is selectively removable from the adhesive layer, and the adhesive layer is adherable to skin.

### BRIEF DESCRIPTION OF THE DRAWINGS

So that the manner in which the features and advantages of the system, products, and/or method so of the present disclosure may be understood in more detail, a more particular description briefly summarized above may be had by reference to the embodiments thereof which are illustrated in the appended drawings that form a part of this specification. It is to be noted, however, that the drawings illustrate only various exemplary embodiments and are therefore not to be considered limiting of the disclosed concepts as it may include other effective embodiments as well.

FIG. 1 depicts two anti-chafing pads being worn by a user, in accordance with certain aspects of the present disclosure;

FIG. 2A is a top view of an anti-chafing pad, in accordance with certain aspects of the present disclosure;

FIG. 2B is a bottom view of the anti-chafing pad of FIG. 2A;

FIG. 2C is a bottom view of the anti-chafing pad of FIG. 2A after removal of a cover layer;

FIG. 2D is a side view of the anti-chafing pad of FIG. 2A;

FIG. 2E is a side view of the anti-chafing pad of FIG. 2A after removal of the cover layer;

FIG. 2F is a side view of the anti-chafing pad of FIG. 2E after adherence to skin;

FIG. 3A is a sectional view of the two thighs of FIG. 1, along line A-A, with each thigh exhibiting “thigh gap” and having an anti-chafing pad applied thereto;

FIG. 3B depicts the two thighs of FIG. 3A, but not exhibiting “thigh gap”;

FIG. 3C depicts the two thighs of FIG. 3B, but without an anti-chafing pad applied thereto;

FIGS. 4A and 4B depict rectangular-shaped pads; and

FIGS. 5A and 5B depict square-shaped pads.

Apparatus, products, and methods according to present disclosure will now be described more fully with reference to the accompanying drawings, which illustrate various exemplary embodiments. Concepts according to the present disclosure may, however, be embodied in many different forms and should not be construed as being limited by the illustrated embodiments set forth herein. Rather, these embodiments are provided so that this disclosure will be thorough as well as complete and will fully convey the scope of the various concepts to those skilled in the art and the best and preferred modes of practice.

### DETAILED DESCRIPTION

Certain aspects of the present disclosure include apparatus, including anti-chafing pads, and methods of reducing and/or preventing chafing, such as chafing on the inner thighs of a person. While aspects shown and described herein are directed to chafing on the inner thighs, the apparatus and methods disclosed herein are also applicable to other portions of skin, such as between the arm and chest.

Certain aspects of the present disclosure include apparatus and methods configured to help both men and women feel confident enough to wear skirts, dresses, and/or shorts, without experiencing painful rubbing between the legs (i.e., chafing). In some aspects, the apparatus disclosed herein is easy to use and cost efficient. In certain aspects, the apparatus reduces the occurrence of chafing. In some such aspects, the apparatus eliminates the occurrence of chafing, providing users with a chafe-free experience.

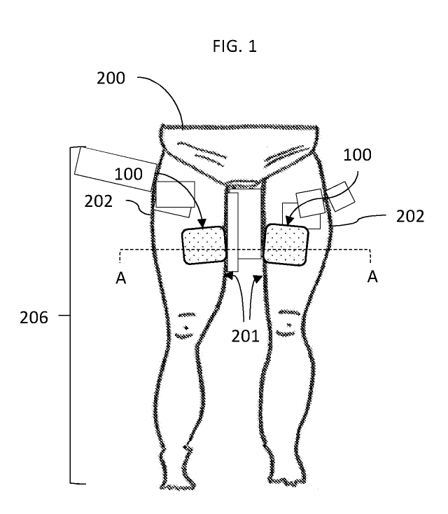

FIG. 1 depicts two anti-chafing pads 100 in accordance with certain aspects of the present disclosure. Each anti-chafing pad 100 is applied to one inner side 201 of one thigh 202 of user 200. Anti-chafing pads 100 are positioned opposite one another, such that the anti-chafing pads 100 are positioned on opposing thighs 202 of user 200. Anti-chafing pads 100 may be positioned anywhere along length 206 of the user's legs where it is expected that contact between the legs may occur during walking or other activities. While shown as including only two pads 100, more than two pads may be used. In some aspects, anti-chafing pads 100 are adhered to the skin of the user.

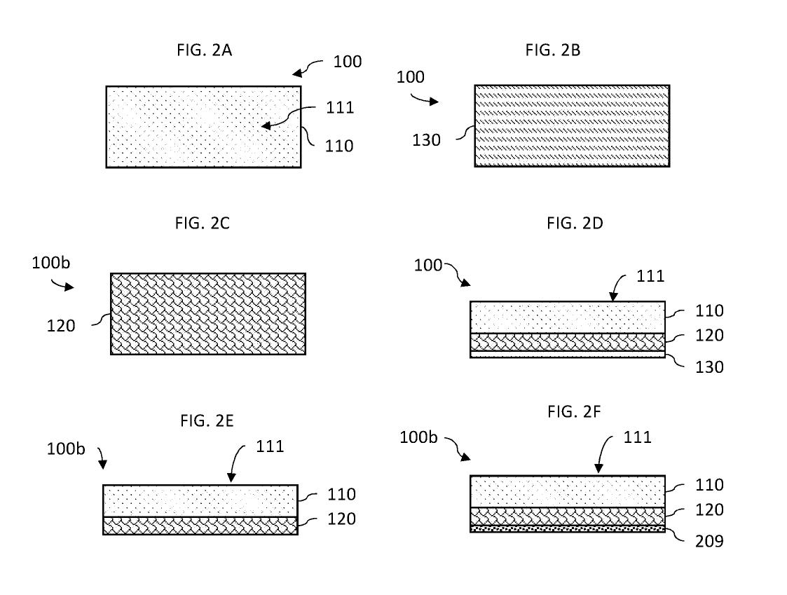

With reference to FIGS. 2A-2F, in certain aspects each anti-chafing pad 100 includes protective layer 110, adhesive layer 120, and cover layer 130. In use, user may remove cover layer 130 from adhesive layer 120 (e.g., by peeling layer 130 from layer 120), and apply adhesive layer 120 to skin 209. In some aspects, protective layer 110 is the topmost layer of pad 100, and pad 100 does not include any additional layers above protective layer 110. In some aspects, cover layer 130, when used and prior to being removed, is the bottommost layer of pad 100, and pad 100 does not include any additional layers below cover layer 130. In some aspects, adhesive layer 120, when cover layer 130 is not used or after cover layer 130 has been removed, is the bottommost layer of pad 100, and pad 100 does not include any additional layers below adhesive layer 120. In some aspects, adhesive layer 120 is positioned directly below protective layer 110 and is in direct contact therewith, such that no layer or other material or component resides between adhesive layer 120 and protective layer 110. In some such aspects, where cover layer 130 is used, cover layer 130 is directly below adhesive layer 120 and is in direct contact therewith, such that no layer or other material or component resides between adhesive layer 120 and cover layer 130. In some aspects, pad 100 is includes at most two layers, including adhesive layer 120 and protective layer 110. In other aspects, pad 100 is includes at most three layers, including cover layer 130, adhesive layer 120, and protective layer 110.

Protective layer 110 may be made of a material configured to prevent chafing and optionally provide cushioning between the thighs 202. For example, protective layer may be at least partially composed of moleskin or another cotton or cotton-based material. In some aspects, protective layer 110 may have an adhesive layer incorporated thereon. For example, protective layer 110 may include latex-free moleskin with an adhesive backing. Protective layer 110 may include cotton that allows for easy gliding movements when worn by a user. Protective layer 110 may be or include clotting gauze, adhesive cushion moleskin, sterilized adhesive bandage, first aid plaster, a waterproof material, physio tape, and/or kinesiology tape or another elastic stretch material.

When worn by a user, outer surface 111 of protective layer 110 forms an engagement surface, such that outer surface 111 engages with opposing engagement surfaces (e.g., opposing surfaces of skin of the opposite thigh or an opposing engagement surface of another pad 100 on the opposite thigh). As such, the skin 209 underlying pad 100b is protected from engagement with the opposing engagement surface. Protective layer 110 may cushion and soften impact and/or frictional engagement with opposing engagement surfaces.

Adhesive cover layer 130 may be a layer of paper or plastic that covers adhesive layer 120 prior to use. When adhering anti-chafing pads 100 to the skin, users remove adhesive cover layer 130 from anti-chafing pads 100 to expose adhesive layer 120. Adhesive layer 120 may then be applied to the desired area of skin. In some aspects, adhesive layer 120 includes a pressure-sensitive adhesive, such that a user may place anti-chafing pads 100 onto the skin and apply pressure thereto to activate the pressure-sensitive adhesive, adhering anti-chafing pads 100 to the skin. In certain aspects, the adhesive is tacky enough to adhere to the skin, but not so tacky that it is difficult to remove from the skin or causes the user to experience pain upon removal of anti-chafing pads 100. In some aspects, the adhesive functions to ensure that anti-chafing pads 100 stays in place on the skin during use, preventing portions of the skin of the inner thighs 202 from touching and allowing the user's legs to move freely and comfortably. In some aspects, anti-chafing pads 100 are in the form of a sticker. Adhesives suitable for use in adhering anti-chafing pads 100 onto the skin include, but are not limited to, vinyl resin-based adhesives, such as acrylate, methacrylate, and epoxy diacrylate based adhesives; adhesive acrylic glue; silicone-based adhesives; and/or gel-based adhesives.

Anti-chafing pads 100 may be configured (e.g., sized and shaped) such that, when adhered to the skin, anti-chafing pads 100 do not interfere with a user's leg movement, allowing the user to freely and comfortably move their legs.

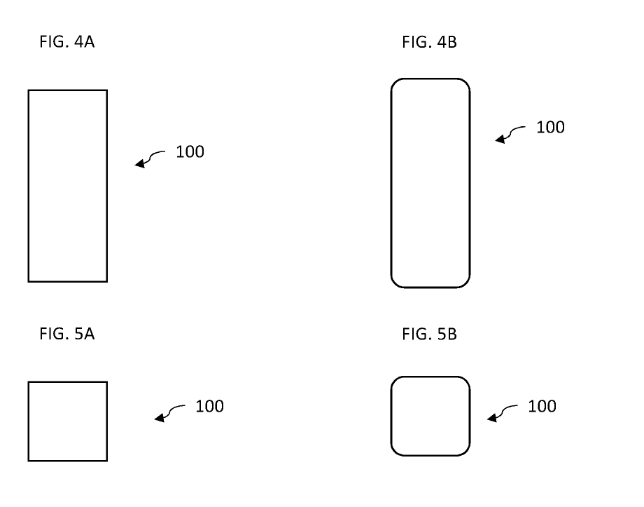

In some aspects, anti-chafing pads 100 are rectangular, square, or other shaped pads or stickers that a user can adhere to the inside of the thighs (or other portion of skin). Pads 100 may have the shape of a rectangle with sharp or rounded corners, as shown in FIGS. 4A and 4B respectively, or may have the shape of a square with sharp or rounded corners, as shown in FIGS. 5A and 5B respectively. Pads 100 may be shaped and sized to accommodate different sized thighs. In some aspects, the size of pads 100 may be customizable, such as by cutting the pads 100 to fit the user (e.g., using scissors). Some exemplary sizes of pads 100 include 9″ by 4″, 9″ by 9″, and 4″ by 4″. However, pads 100 may be any size, including smaller than 4″ by 4″ and larger than 9″ by 9″.

In some aspects, the inside surface, outside surface, or an entirety of each anti-chafing pad 100 is made of silicone, foam (e.g., a polymer foam), fabric, or another material capable of sticking to the user's skin and providing comfort.

In some aspects, anti-chafing pads 100 are disposable, such that anti-chafing pads 100 are designed to be disposed of after one use. In other aspects, anti-chafing pads 100 are usable more than once, such that anti-chafing pads 100 may be applied to the skin and used on a first occasion, taken off of the skin, optionally washed, and subsequently applied to the skin and used on a second occasion.

In some aspects, by preventing or reducing direct skin-to-skin contact between two portions of the body (e.g., between two thighs), anti-chafing pads 100 reduce or eliminate chafing. As such, the use of anti-chafing pads 100 reduce or eliminate the annoyance, pain, rash, redness, and/or irritation that results from chafing.

In some aspects, anti-chafing pads 100, when worn, do not constrict the legs of the user, do not cause discoloration (e.g., whitening) of clothes, do not lose effectiveness at reducing chafing over time, remain in place on the inner thigh 202 (or other portion of skin) during the entirety of use, or combinations thereof. Anti-chafing pads 100 may remain in place and maintain effectiveness at reducing chafing for a period of at least 8 hours, or at least 12 hours, or at least 18 hours, or at least 24 hours.

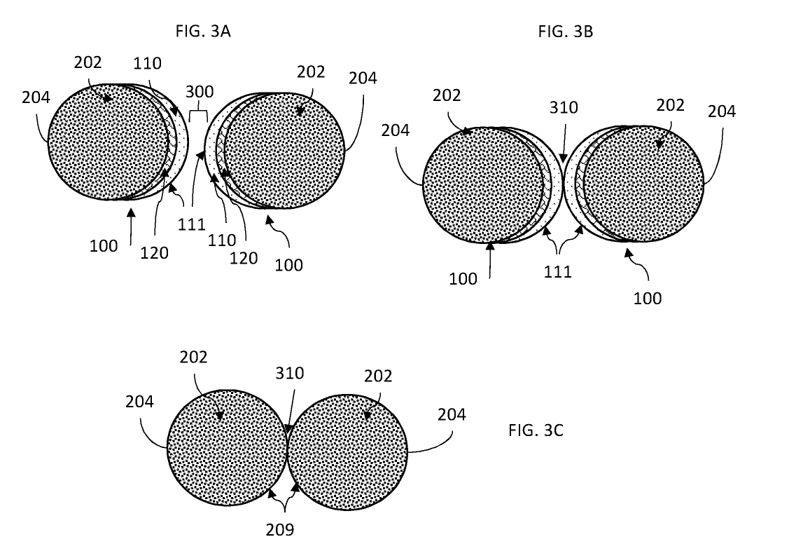

With reference to FIG. 3A, in some aspects each anti-chafing pad 100, when worn on thigh 202, extends only partially about circumference 204 of thigh 202, such that each anti-chafing pad 100 does not fully circumferentially surround thigh 202. In some aspects, the anti-chafing pads 100, when worn on thighs 202, extend only partially along length 206 of thigh 202, as shown in FIG. 1. In FIG. 3A, thighs 202 are spaced apart, at least partially exhibiting thigh-gap 300. Adhesive layer 120 may be, for example, an about 4″ wide gel adhesive layer that is attached to skin of thigh 202. Outer surfaces 111 of layer 110 are positioned such that if thighs 202 move close enough together, outer surfaces 111 will engage one another rather than the skin of the thighs 202 engaging. During use, pads 100 may be positioned on the inner thighs of the user, such that pads 100 may be concealed from view. In some aspects, pad only attached to skin of the user, and does not attached to clothing.

In FIG. 3B, thighs 202 are not fully spaced apart, such that a thigh-gap is not exhibited. Rather than exhibiting a thigh gap, thighs 202 in FIG. 3B are at least partially engaged. With thighs 202 at least partially engaged, outer surfaces 111 of pads 100 are at least partially engaged at engagement point or plane 310. By selective and strategic placement of pads 100 on portions of thighs 202 expected to engage, users may provide cushioning at, along and/or within engagement point or plane 310, such that the skin at these locations is not subjected to frictional engagement. During use, outer surfaces 111 of protective layers 110 slidingly engage one another, rubbing against one another as the thighs 202 move (e.g., during walking). Protective layers 110 may be at least partially composed of a material such that protective layers 110 do not stick to one another, but smoothly slide relative to one another when frictionally engaged. In some aspects, outer surfaces 111 are at least partially composed of a material that is different than the remainder of protective layer 110, such as a material that provides a low enough coefficient of friction between outer surfaces 111 that outer surfaces slide past one another during frictional engagement without a substantial degree of sticking therebetween. For example, such a material may form a smooth or slick layer or surface of protective layer 110, allowing for relatively smooth, gliding between two pads 100 in contact and in motion relative to one another. In some aspects, the outer surface 111 of protective layer is or includes a waterproof or water resistance material. In some aspects, each anti-chafing pad 100 is sufficiently sized such that it covers an entirety of skin surface area that would otherwise be subjected to chafing For example, and without limitation, each anti-chafing pad 100 may be have a surface area of about 2 in², or about 3 in², or about 4 in², or about 5 in², or about 6 in².

FIG. 3C depicts thighs 202 that do not exhibit a thigh gap and that are frictionally engaged along engagement point or plane 310. As thighs 202 in FIG. 3C do not have anti-chafing pads adhered thereon, skin surfaces 209 of thighs 202 are subjected to frictional engagement with one another, which causes irritation thereof at engagement point or plane 310.

Certain aspects of the present disclosure include methods of preventing chafing, including methods of using the anti-chafing pads disclosed herein. The method includes applying (e.g., adhering) anti-chafing pads to the skin (e.g., the inner thighs). Once the anti-chafing pads are applied to the skin, the user may wear any desired clothing item over the anti-chafing pads (e.g., a skirt, dress, or shorts). With the anti-chafing pads applied to the skin, the user may walk, run, or otherwise move about comfortably and freely, without experiencing chafing, or at least experiencing less chafing than would otherwise occur if the user were not wearing the anti-chafing pads.

In some aspects, the method includes applying two anti-chafing pads positioned opposite one another, such that each anti-chafing pad is positioned on one thigh.

After use, the method includes removing the anti-chafing pads from the skin. In some aspects, the method includes disposing of the used anti-chafing pads after the first use thereof. In other aspects, the method includes reusing the anti-chafing pads after the first use, optionally with intermediate washing thereof.

In some aspects, the method results in a reduction or elimination of chafing, thus reducing or eliminating the annoyance, pain, rash, redness, and/or irritation that results from chafing.

In some aspects, the pads disclosed herein do not include spandex, thigh bands, lace material, or combinations thereof.

In some aspects, the method disclosed herein does not include the application of deodorant, body powder, glide cream, or combinations thereof to the skin. In some aspects, the pads are not placed over a wound or sore. In some aspects, the pads are not placed over a portion of the body to support a dressing. In some such aspects, a wound dressing is not positioned between the pad and the user's skin. In some aspects, the pads do not and are not used to support a splint, are not used in conjunction with a splint, and/or are not used to restrict the movement of a part of the body. In some aspects, the pads are not used to reduce swelling and/or are not used to provide support to a sprained portion of the body. In some aspects, the portion of skin positioned below the pad, when in use, is not wounded, and the pad does not restrict the movement thereof.

Although the present embodiments and advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An anti-chafing pad, the anti-chafing pad comprising:
a protective layer; and
an adhesive layer on the protective layer, wherein the adhesive layer is adherable to skin.

2. The anti-chafing pad of claim 1, wherein the adhesive layer includes a gel adhesive.

3. The anti-chafing pad of claim 1, wherein the adhesive layer includes a vinyl resin adhesive or a silicone adhesive.

4. The anti-chafing pad of claim 3, wherein the vinyl resin adhesive is an acrylate adhesive, a methacrylate adhesive, or an epoxy diacrylate adhesive.

5. The anti-chafing pad of claim 1, wherein the adhesive layer includes a pressure-sensitive adhesive.

6. The anti-chafing pad of claim 1, wherein the protective layer includes cotton.

7. The anti-chafing pad of claim 1, further comprising a cover layer disposed on the adhesive layer, wherein the adhesive layer is positioned between the cover layer and the protective layer, and wherein the cover layer is selectively removable from the adhesive layer.

8. The anti-chafing pad of claim 1, wherein the anti-chafing pad is washable and reusable.

9. The anti-chafing pad of claim 1, wherein the anti-chafing pad, when worn on the skin, prevents chafing of the skin.

10. The anti-chafing pad of claim 1, wherein, when worn on a thigh of a user, the anti-chafing pad extends only partially about a circumference of the thigh.

11. The anti-chafing pad of claim 1, wherein, when worn on a thigh of a user, the anti-chafing pad extends only partially along a length of the thigh.

12. An anti-chafing pad, the anti-chafing pad comprising:
a protective layer;
an adhesive layer on the protective layer, wherein the adhesive layer is adherable to skin; and
a cover layer disposed on the adhesive layer, wherein the adhesive layer is positioned between the cover layer and the protective layer, and wherein the cover layer is selectively removable from the adhesive layer;
wherein the anti-chafing pad, when worn on skin, prevents or reduces chafing of the skin.

13. A method of preventing or reducing chafing on skin of a user, the method comprising:
providing first and second anti-chafing pads, wherein each anti-chafing pad includes a protective layer, an adhesive layer on the protective layer, and a cover layer disposed on the adhesive layer, wherein the adhesive layer is positioned between the cover layer and the protective layer, wherein the cover layer is selectively removable from the adhesive layer, and wherein the adhesive layer is adherable to skin.

14. The method of claim 13, further comprising:
removing the cover layer of the first anti-chafing pad and adhering the adhesive layer of the first anti-chafing pad onto a first portion of skin of the user;
removing the cover layer of the second anti-chafing pad and adhering the adhesive layer the second anti-chafing pad onto a second portion of skin of the user;
wherein the first and second anti-chafing pads are positioned on the skin of the user such that when the user moves, the protective layers of the first and second anti-chafing pads at least partially contact one another.

15. The method of claim 14, wherein the first and second anti-chafing pads are adhered onto opposing inner thighs of the user such that the first and second anti-chafing pads are positioned opposite one another on the opposing inner thighs of the user.

16. The method of claim 14, further comprising wearing a desired clothing item over the anti-chafing pads.

17. The method of claim 14, wherein the anti-chafing pads, when applied to the skin, allow the user to move without experiencing chafing or at least experiencing less chafing than would otherwise occur if the user were not wearing the anti-chafing pads.

18. The method of claim 14, further comprising, after use of the anti-chafing pads, removing the anti-chafing pads from the skin and disposing of the removed anti-chafing pads, or washing and reusing the anti-chafing pads.

19. The method of claim 14, wherein the anti-chafing pads are applied on thighs of the user such that the anti-chafing pads extend only partially about a circumference of the thighs.

20. The method of claim 14, wherein the anti-chafing pads are applied on thighs of the user such that the anti-chafing pads extend only partially along a length of the thighs.
